(19) United States
(12) Patent Application Publication
(10) Pub. No.: US 2021/0007683 A1
(43) Pub. Date: Jan. 14, 2021

(54) LASER-BASED LOCATOR USED IN COMPUTED TOMOGRAPHY

(71) Applicant: Innostar Service, Inc., Taichung (TW)

(72) Inventors: Meng-Chih WU, Taichung (TW); Hsien-Tang TU, Taichung (TW); Tzu-Ching WU, Taichung (TW)

(21) Appl. No.: 16/504,346

(22) Filed: Jul. 8, 2019

(51) Int. Cl. A61B 6/04; A61B 6/03

(52) U.S. Cl. CPC A61B 6/0492; A61B 6/03

(57) ABSTRACT

A laser-based locator includes a column, an axle, a rectilinear track, a sliding element and a laser source. The axle is supported by the column. The rectilinear track is connected to the axle and rotatable relative to the column between an original position and a lifted position. The rectilinear track extends parallel to the column in the original position. A length of the rectilinear track intersects a height of the column in the lifted position. The sliding element is supported on the rectilinear track so that the sliding element is reciprocated along the rectilinear track. The laser source is rotationally connected to the sliding element and operable to emit a laser.

### BACKGROUND OF INVENTION

### 1. Field of Invention

The present invention relates to computed tomography and, more particularly, to a laser-based locator used in computed tomography.

### 2. Related Prior Art

In computed tomography (“CT”), X-ray is used to scan a human body to obtain data related to the human body before the data is processed by a calculator to provide images at various layers of the human body. CT is often used in diagnosis of diseases for advantages such as high precision of the data, high resolution of the images, short time taken to complete the scan, and easy access. However, it is quite troublesome for a user, even an experienced one, to locate a spot on the human body corresponding to a point in the human body where scan is needed.

Apparatuses have been devised to solve the above-mentioned problem. For example, as disclosed in WO2006/125605, FIG. 3, a computed tomographic machine includes a radiation source 10 supported on a semi-circular track (or “carrier”) 9 defined by a constant radius 11. The radiation source 10 can be reciprocated along the hemi-circular track 9. If necessary, the radiation source 10 can emit a laser 1 to cast a light spot at a point on an object corresponding to a point in the object where scan is needed. However, the hemi-circular track 9, which is defined by the constant radius 11, limits the movement of the radiation source 10 to a small range, thereby rendering it difficult to cast the light spot exactly at the point on the object corresponding to the point in the object where scan is needed.

Alternatively, referring to FIG. 4a of WO2006/125605, the radiation source 10 is connected to a robot. The robot includes sections 9a connected to one another by joints 9C so that the sections 9a can pivot relative to one another. A stepper motor 15 is connected to one of the sections 9a or one of the joints 9c to move the radiation source 10 so that the radiation source 10 can emit a laser 1 to cast a light spot at a point on an object corresponding to a point in the object. However, with the use of the robot, the movement of the radiation source 10 is limited to a small range, thereby rendering it difficult to cast the light spot exactly at the point on the object corresponding to the point in the object where scan is needed.

The present invention is therefore intended to obviate or at least alleviate the problems encountered in prior art.

### SUMMARY OF INVENTION

It is the primary objective of the present invention to provide a computed tomographic machine with an effective laser-based locator.

To achieve the foregoing objective, the laser-based locator includes a column, an axle, a rectilinear track, a sliding element and a laser source. The axle is supported by the column. The rectilinear track is connected to the axle and rotatable relative to the column between an original position and a lifted position. The rectilinear track extends parallel to the column in the original position. A length of the rectilinear track intersects a height of the column in the lifted position. The sliding element is supported on the rectilinear track so that the sliding element is reciprocated along the rectilinear track. The laser source is rotationally connected to the sliding element and operable to emit a laser.

Other objectives, advantages and features of the present invention will be apparent from the following description referring to the attached drawings.

### BRIEF DESCRIPTION OF DRAWINGS

The present invention will be described via detailed illustration of two embodiments referring to the drawings wherein:

FIG. 1 is a perspective view of a laser-based locator according to the first embodiment of the present invention;

FIG. 2 is a top view of the laser-based locator shown in FIG. 1 without a cover;

FIG. 3 is a perspective view of two laser modules of the laser-based locator shown in FIG. 2;

FIG. 4 is a cross-sectional view of one of the laser modules shown in FIG. 3;

FIG. 5 is a perspective view of a light emitter of the laser-based locater shown in FIG. 1;

FIG. 6 is a perspective view of the laser-based locater shown in FIG. 1 used in computed tomography; and

FIG. 7 is a perspective view of a laser-based locator according to the second embodiment of the present invention.

### DETAILED DESCRIPTION OF EMBODIMENTS

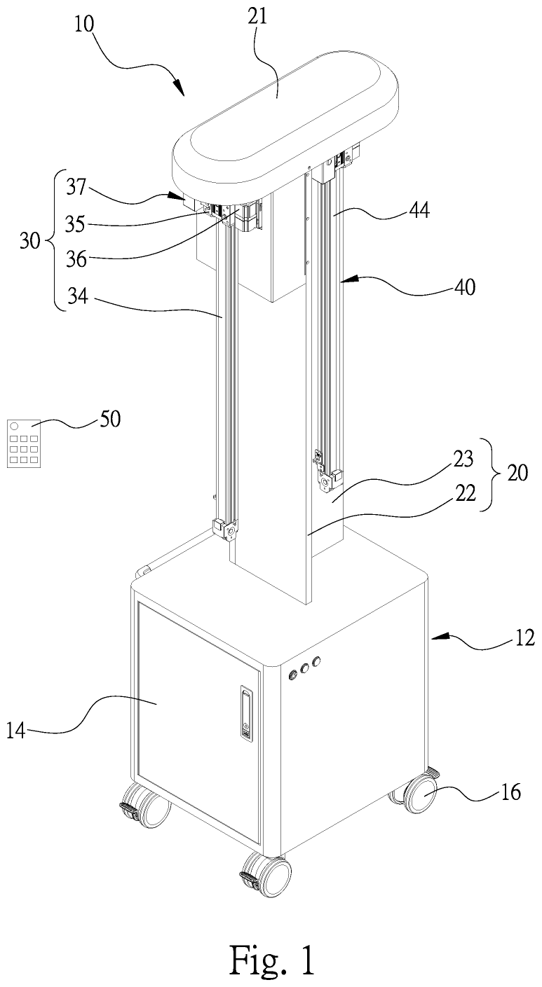

Referring to FIG. 1, a laser-based locator 10 includes a base 12, a column 20, two laser modules 30 and 40 and a remote controller 50 according to a first embodiment of the present invention. The remote controller 50 is operable to control the laser-based locator 10 at a distance.

The base 12 includes a box 14 and casters 16 connected to a lower face of the box 14. The box 14 provides electricity to the laser-based locator 10. The casters 16 are switchable between an unlocked mode and a locked mode. In the unlocked mode, the casers 16 allow the laser-based locator 10 to move. In the locked mode, the casters 16 keep the laser-based locator 10 in position.

The column 20 includes a canopy 21 supported on two vertical plates 22 and 23 and two shields 24 and 25. The vertical plates 22 and 23 are supported on an upper face of the box 14, thereby holding the canopy 21 at a desired height from a floor that supports the casters 16 of the base 12 of the laser-based locator 10. The shield 24 is secured to the vertical plate 22. The shield 25 is secured to the vertical plate 23.

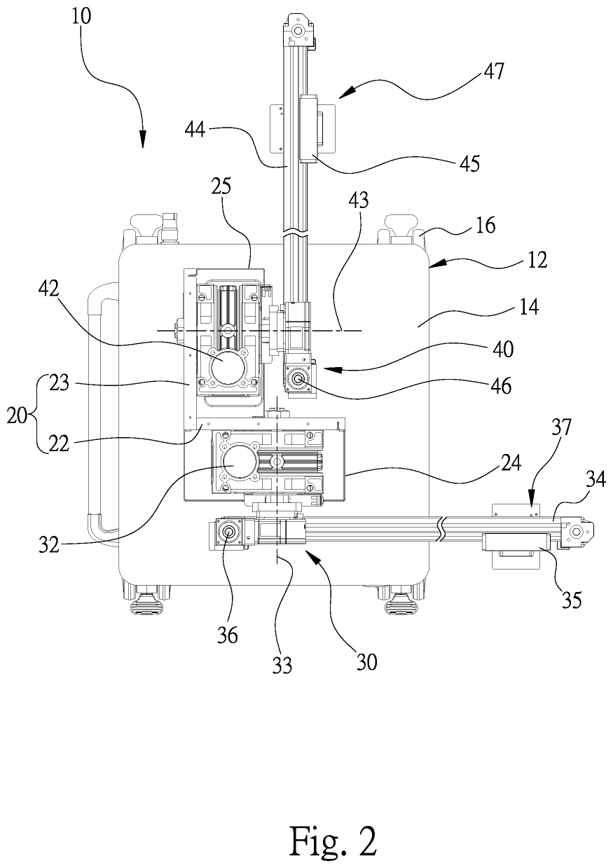

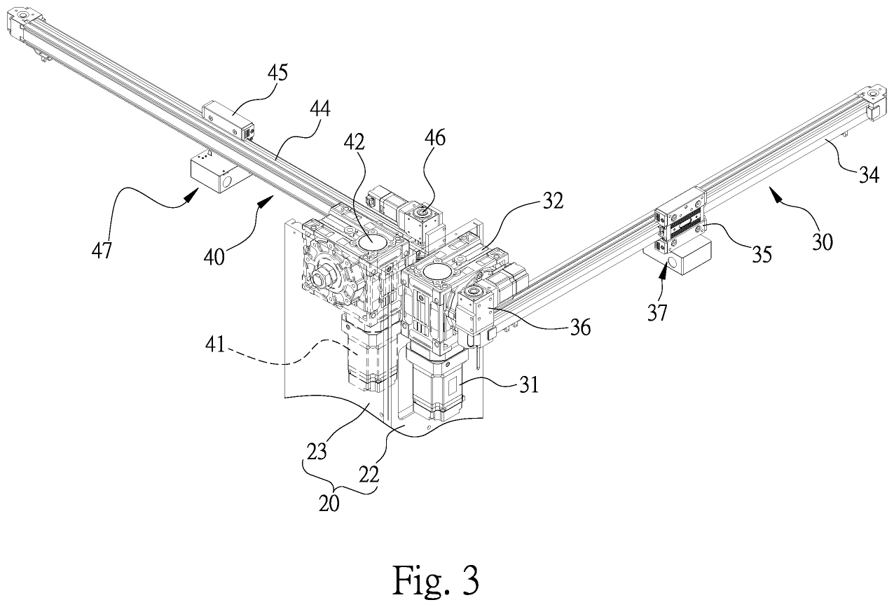

Referring to FIGS. 1 through 3, the laser module 30 includes a motor 31, a transmission 32, an axle 33, a rectilinear track 34, a sliding element 35, a motor 36 and a light emitter 37. The motor 31 is connected to the transmission 32. The transmission 32 is protected by the canopy 21 and the shield 24. The transmission 32 is connected to the rectilinear track 34 that is located on a side of the vertical plate 22. Via the transmission 32, the motor 31 is operable to pivot the rectilinear track 34 about the axle 33 between an original position and a lifted position relative to the vertical plate 22 (or the column 20). In the original position, the rectilinear track 34 extends downwards, parallel to the column 20. In the lifted position, the rectilinear track 34 extends horizontally.

The transmission 32 is preferably a gear box secured to the vertical plate 22. In another embodiment, the transmission 32 can be a belt-and-pulley combination, a chain-and-sprocket combination or any other proper mechanical mechanism for transmitting torque.

The sliding element 35 is movably supported on the rectilinear track 34 and normally located in the vicinity of the canopy 21. The motor 36 is located in the vicinity of the rectilinear track 34 and connected to the sliding element 35. Thus, the motor 36 is operable to reciprocate the sliding element 35 along the rectilinear track 34. The light emitter 37 is supported on the sliding element 35.

Similarly, the laser module 40 includes a motor 41, a transmission 42, an axle 43, a rectilinear track 44, a sliding element 45, a motor 46 and a light emitter 47. The motor 41 and the transmission 42 are secured to the vertical plate 23. The transmission 42 is protected by the canopy 21 and the shield 25. The motor 41 is connected to the transmission 42 that is connected to the rectilinear track 44. Via the transmission 42, the motor 41 is operable to pivot the rectilinear track 44 about the axle 43 relative to the vertical plate 23 (or the column 20) between an original position and a lifted position. The rectilinear track 44 is located on a side of the vertical plate 23. In an original position, the rectilinear track 44 extends downwards. In the lifted position, the rectilinear track 44 extends horizontally.

The rectilinear tracks 34 and 44 extend parallel to each other when they are in the original positions, respectively. A length of the rectilinear track 44 intersects a length of the rectilinear track 34 when the rectilinear tracks 34 and 44 are in the lifted positions, respectively.

The rectilinear track 44 supports the sliding element 45. The motor 46 is located in the vicinity of the rectilinear track 44. The motor 46 is connected to the sliding element 45. Thus, the motor 46 is operable to move the sliding element 45 to and fro along the rectilinear track 44. The light emitter 47 is supported on the sliding element 45 so that the light emitter 47 is movable with the sliding element 45.

It should be noted that the vertical plates 22 and 23 are joined so that the axles 33 and 43 intersect each other.

The laser module 40 is identical to the first laser module 30 in structure. In specific, the light emitters 37 and 47 are identical to each other in structure. Hence, the following description will be given to the light emitter 37 only for briefness.

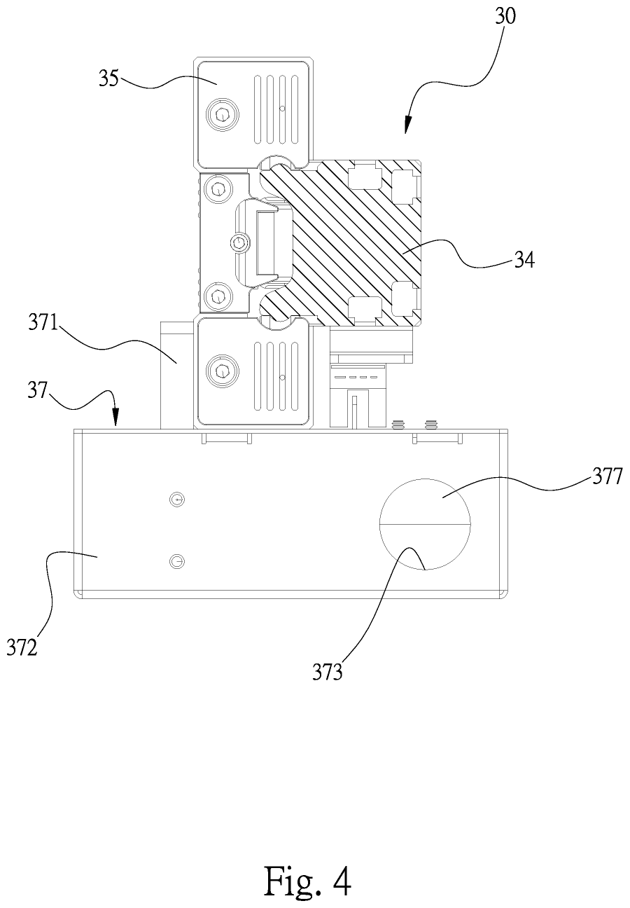

Referring to FIG. 4, the light emitter 37 includes a connective portion 371 on an external face. The connective portion 371 is detachably connected to the sliding element 35 and a shell 372. The shell 372 provides a vent 373. A motor 377 that is located in the light emitter 37 is visible through the vent 373. Thus, the shell 372 protects elements in the light emitter 37, and heat produced by the elements in the light emitter 37 is removed from the light emitter 37 via the vent 373.

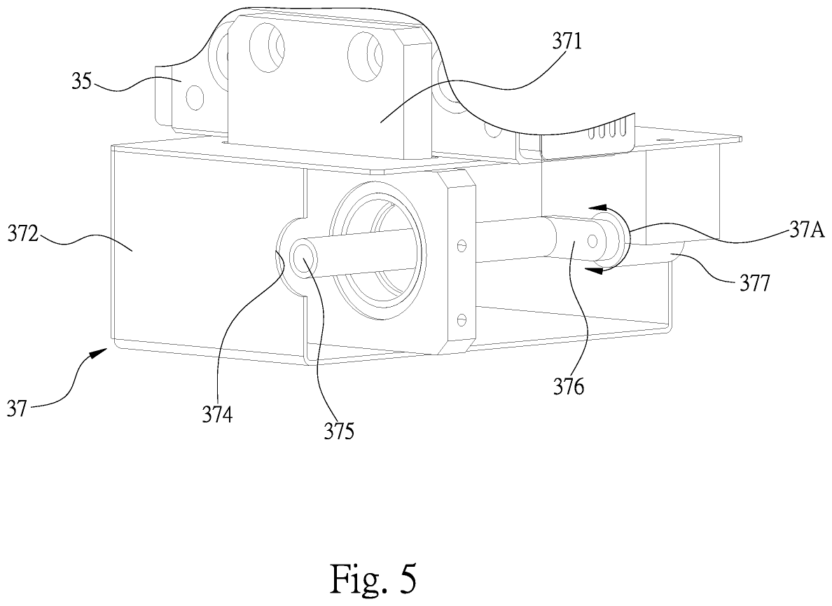

Referring to FIG. 5, the interior of the light emitter 37 will be described. A laser source 375 is connected to the motor 377 via a mechanical structure 376. The motor 377 can be actuated to spin the mechanical structure 376 in a sense of direction represented by an arrow head 37A, thereby rotating the laser source 375 relative to the shell 372 (or the sliding element 35). The laser source 375 emits a laser that goes out of the shell 372 via a window 374.

In the first embodiment, the mechanical structure 376 is a rod. In another embodiment, the mechanical structure 376 can be a gear train, a belt-and-pulley combination, a chain-and-sprocket combination or any other proper mechanical mechanism.

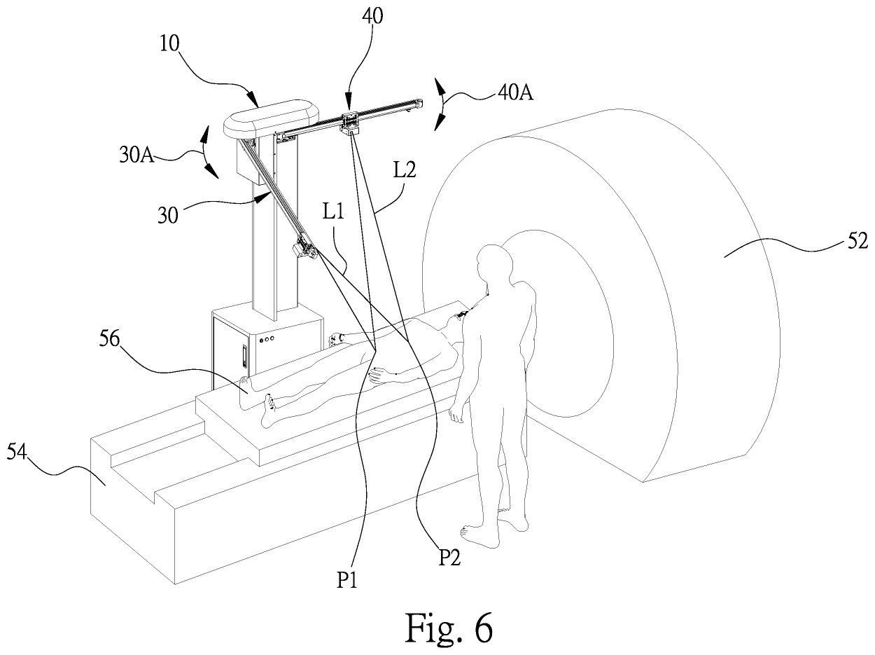

Referring to FIG. 6, in operation, the laser-based locator 10 is located in the vicinity of a computed tomographic machine 52 and a check bench 54. According to scanned images or other data, or controlled by the remote controller, the rectilinear track 34 of the laser module 30 is rotated to a predetermined angle in a sense of direction represented by an arrow head 30A before it is operated to emit a laser L1 to the check bench 54. The laser L1 produces a light spot on a checked body 56 on check bench 54 to provide an anchor point P1. The anchor point P1 is a reference point on the checked body 56 corresponding to a correct point in the checked body 56. Of course, the laser L1 emitted from the laser module 30 can be pointed at another point in the checked body 56, thereby providing an anchor point P2 on the checked body 56.

For precision, the rectilinear track 44 of the second laser module 40 is lifted to a predetermined angle in a sense of direction represented by an arrow head 40A before it is operated to emit a laser L2 to the check bench 54. The laser L2 produces anchor point P1 or P2 on the checked body 56 corresponding to a correct point in the checked body 56.

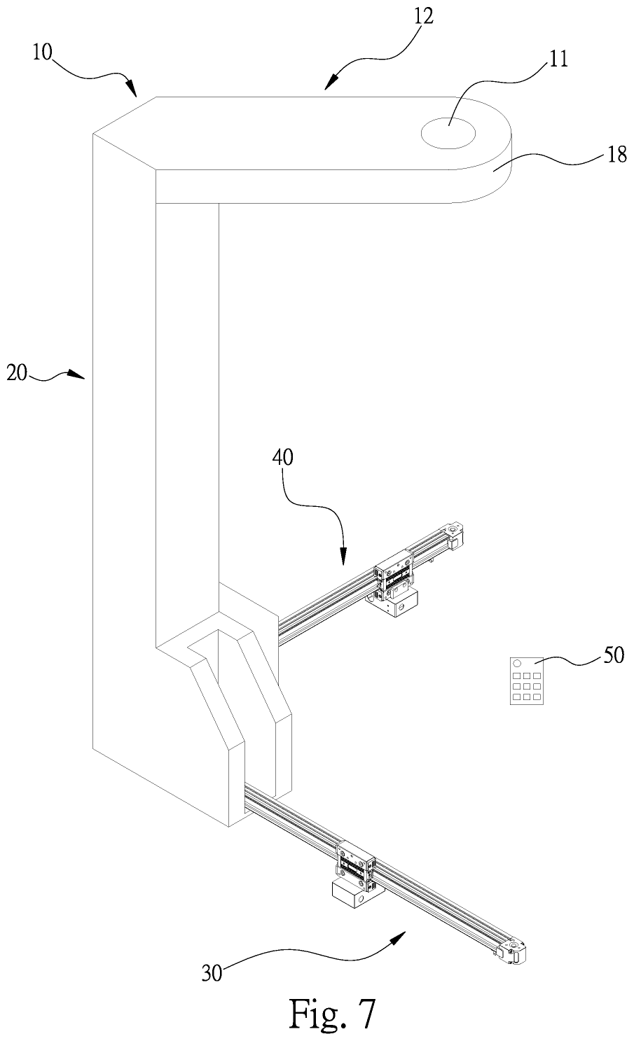

Referring to FIG. 7, a laser-based locator 10 according to a second embodiment of the present invention is shown. The second embodiment is identical to the first embodiment except for including a connective element 18 in the form of a plate instead of the base 12. An upper end of the column 20 is connected to a lower face of the connective element 18. In operation, the connective element 18 is connected to a ceiling of a building (not shown) so that the column 20 dangles from the connective element 18, which is connected to the ceiling.

The connective element 18 and the column 20 are hollow to contain wires related to the laser modules 30 and 40. These wires extend out of the connective element 18 through an aperture 11. These wires can be electrically connected to the mains power.

Each of the laser modules 30 and 40 is partially covered by the column 20. However, the column 20 does not interfere with the use of the remote controller 50 to control the laser module 30 or 40.

The present invention has been described via the illustration of the embodiments. Those skilled in the art can derive variations from the embodiments without departing from the scope of the present invention. Therefore, the embodiments shall not limit the scope of the present invention defined in the claims.

1. A laser-based locator comprising:
a column;
an axle supported by the column;
a rectilinear track rotatable relative to the column about the axle between an original position and a lifted position, wherein the rectilinear track extends parallel to the column in the original position, and a length of the rectilinear track intersects a height of the column in the lifted position;
a sliding element movable to and fro along the rectilinear track; and
a laser source rotationally connected to the sliding element and operable to emit a laser.

2. The laser-based locator according to claim 1, comprising a transmission connected to the axle and operable to pivot the rectilinear track relative to the column about the axle.

3. The laser-based locator according to claim 2, comprising a motor for driving the transmission.

4. The laser-based locator according to claim 3, comprising a base for supporting wherein the column.

5. The laser-based locator according to claim 4, wherein the base comprises a box for supporting the column on a floor.

6. The laser-based locator according to claim 5, wherein the base comprises casters connected to the box.

7. The laser-based locator according to claim 3, comprising a connective element formed with a lower face connected to an upper end of the column, wherein the connective element is connected to a ceiling in operation.

8. A laser-based locator comprising:
a column;
two axles connected to the column;
two rectilinear tracks each of which is rotatable about one of the axles between an original position and a lifted position, wherein the rectilinear tracks extend parallel to each other in the original positions, and lengths of the rectilinear tracks intersect each other in the lifted positions;
two sliding elements each of which is movable to and fro along one of the rectilinear tracks; and
two laser sources each of which is rotationally connected to one of the sliding elements and used to emit a laser.

9. The laser-based locator according to claim 8, wherein the column comprises two verticals plate connected to each other at an angle of 90 degrees.

10. The laser-based locator according to claim 9, comprising:
two transmissions each of which is connected to one of the axles and is supported on one of the vertical plates; and
two motors each of which drives one of the rectilinear tracks relative to the column via the axle of one of the transmissions.
